(19) United States
(12) Patent Application Publication
(10) Pub. No.: US 2023/0165994 A1
(43) Pub. Date: Jun. 1, 2023

(54) Effervescent Tablet and Composition for Masking Toilet Odors and Method of Use Thereof

(71) Applicant: Crystal Andersen, Payson, UT (US)

(72) Inventor: Crystal Andersen, Payson, UT (US)

(21) Appl. No.: 17/697,066

(22) Filed: Mar. 17, 2022

Related U.S. Application Data
(60) Provisional application No. 63/283,908, filed on Nov. 29, 2021.

(51) Int. Cl. A61L 9/013; A61L 9/012; E03D 9/00

(52) U.S. Cl. CPC A61L 9/013; A61L 9/012; E03D 9/005; A61L 2209/21

(57) ABSTRACT

The present invention relates to a small, effervescent tablet for disguising bathroom odors. The tablet includes sodium bicarbonate, a mild acid and natural or essential oils. The tablet is wrapped in a moisture proof recyclable foil that can be torn along a perforation line to use the tablet. The tablet is simply dropped into a plumbing fixture and when the tablet is diluted in water, an effervescence is produced releasing carbon dioxide and the aroma of the natural oil is released to mask the unwanted odor. The packaging of an individual tablet is compact allowing a user to store and conceal the tablet in purses, waistbands, clothing, wallets, pockets, boots, and even in phone cases.

### CROSS-REFERENCE TO RELATED APPLICATION

The present application claims priority to, and the benefit of, U.S. Provisional Application No. 63/283,908, which was filed on Nov. 29, 2021, and is incorporated herein by reference in its entirety.

### FIELD OF THE INVENTION

The present invention relates generally to odor masking devices. More specifically, the present invention relates to an effervescent tablet designed to be dropped in a toilet bowl or other plumbing fixture for masking unwanted odors arising out of a toilet bowl or plumbing fixture. The effervescent tablet is small and compact in size and can be easily carried in purses, backpacks, satchels and concealed by an individual. The tablet has natural composition and is safe for toilet bowls, plumbing and septic systems. The tablet offers a way to disguise restroom, bathroom, and other plumbing odors in public areas like gyms, schools, cafes, and other public areas. Accordingly, the present disclosure makes specific reference thereto. Nonetheless, it is to be appreciated that aspects of the present invention are also equally applicable to other like applications, devices and methods of manufacture.

### BACKGROUND

By way of background, bathroom odors can be overpowering and embarrassing, especially in public spaces such as gyms, schools, hotels, convention centers, restaurants and other places. Many individuals are reluctant to use a public restroom without the ability to conceal the odors they may leave behind. Also, people are reluctant to use smelly restrooms. Generally, before and after using a restroom, people want to mask the unpleasant smell and therefore, use a variety of methods for removing and masking the odors or other undesirable smells. For example, individuals open windows for natural ventilation, spray an air freshener or use liquid spray products on toilet bowl seat, in the bathroom or other areas where such odor may be encountered. However, these methods may not be effective and some of them require an individual to carry a spray device which may not always be possible for the individual, such as when traveling by air where the amount of liquid that may be carried is limited. The individual is required to carry a bag to carry the device. It is also embarrassing for a person to carry such air fresheners while going to the restroom. In some restrooms, such devices are provided, but people are hesitant to touch and use them because of hygiene issues. Often such air fresheners may be empty, broken or otherwise inoperative. People desire a new product to eliminate bathroom smells and odors before and after use and that can be carried and concealed easily in purses, waistbands, clothing, wallets, pockets, boots, and even in phone cases.

As stated earlier, people face the problem of encountering restroom odors in public areas like gyms, schools, cafes, and other public areas and people may not be carrying spray or other odor-diffusing devices with them. Further, these devices are expensive to purchase and difficult to use. Accordingly, people become reluctant to use such public restrooms and other areas. People desire a specific product that can be used with toilet bowls and plumbing fixtures to eliminate odors before and after use of the restroom.

Many products that are used with toilet bowls and other plumbing, such as, sinks, bidets, septic systems, sump pits and the like for removing odor, are usually acidic in nature and have very low pH. Such products can be detrimental to porcelain fixtures such as, toilet bowls, toilet seats, bidets, sinks and other plumbing bases and therefore, people desire a fragrant product that is safe for plumbing fixtures such as toilet bowls, bidets and septic systems.

Therefore, there exists a long-felt need in the art for an improved solution that can effectively disguise odorous bathrooms. There is also a long-felt need in the art for a fragrance providing device that can be easily used in restrooms, bathrooms, and other areas and specifically with toilet bowls and bidets. Additionally, there is a long-felt need in the art for a fragrant product that can be easily and conveniently carried by an individual in a pocket, sachet, or in a purse. Moreover, there is a long-felt need in the art for a scent product that is safe for toilet bowls, bidets and septic systems. Further, there is a long-felt need in the art for a product that is easy to use and does not leave any residue in the plumbing fixture after use. Finally, there is a long-felt need in the art for a way that easily disguises or conceals restroom odors in public areas like gyms, schools, cafes, hotels, convention centers, restaurants, and other public spaces.

The subject matter disclosed and claimed herein, in one embodiment thereof, comprises an effervescent tablet for eliminating or reducing odorous bathroom smells. The effervescent tablet has a composition comprising sodium bicarbonate, natural oil and vinegar in powder or granule form. The effervescent tablet, when dropped into a toilet bowl, bidet or other plumbing fixture, is diluted to create an effervescence; and the tablet, upon dilution, emits a pleasant and odor masking scent to mask or conceal unwanted odors that may be found in a restroom after use. The tablet is small and discreet and can be highly portable. Preferably, the tablet is coin size, ranging from 0.7 inches to about 1.2 inches with about 0.8 inch to about 1 inch being preferred and can be concealed in purses, waistbands, clothing, wallets, pockets, boots, and even in phone cases.

In this manner, the effervescent tablet of the present invention accomplishes all of the forgoing objectives and provides users with dissolvable wafers for use in bathrooms to eliminate smells and odors. The tablet contains natural ingredients, is easy to use and rapidly dissolves with no greasy build up in toilets, bidets, sinks and other plumbing fixtures. The tablet features a small, compact design which is easy to conceal and carry on the go and can be used in restrooms or other facilities located in public areas.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an effervescent tablet for disguising or concealing odorous bathroom smells. The effervescent tablet includes sodium bicarbonate, natural oil and vinegar in a powder or granule form. The effervescent tablet, when dropped into a toilet, bidet or other plumbing bowl or sink, is diluted to create an effervescence. The tablet, upon dilution, emits a pleasant and odor masking scent.

In yet another embodiment, the weight ratio of vinegar to sodium bicarbonate in the effervescent tablet is less than 0.3 to 1.

In yet another embodiment, the natural oil are essential oils and includes one of lemon oil, lemongrass oil, rosemary oil, lavender oil, tea tree oil, peppermint, spearmint, citrus, and orange oil.

In yet another embodiment, the natural oil comprises about 5%-10% by weight of the total composition of the tablet.

In yet another embodiment, the effervescent tablet is generally circular shaped.

In yet another embodiment, the effervescent tablet is generally rectangular or square shaped.

In yet another embodiment, the effervescent tablet is wrapped in a recyclable wrapper made from a moisture proof foil.

In yet another embodiment, the effervescent tablet is packaged in a single use package which is carried in a purse or a pocket.

In yet another embodiment, the effervescent tablet is packaged and available in a pack of 20 effervescent tablets, 50 effervescent tablets and 200 effervescent tablets.

In yet another embodiment, an effervescent tablet composition for emitting pleasant fragrance that masks unwanted odors arising out of a toilet, bidet, sink or other plumbing bowl is disclosed. The composition includes sodium bicarbonate, natural oil and citric acid. The composition is configured to produce effervescence effects and releases fragrance upon being diluted after exposure to water when the effervescent tablet is dropped in a toilet, bidet, sink or other plumbing bowl. The tablet takes 1-2 minutes to completely dissolve in water.

In yet another embodiment, the pH of the water contained within the plumbing receptacle, after the effervescent tablet is dissolved or diluted, is in the range between 5.5 and 7, thereby further killing bacteria to promote hygiene.

In yet another embodiment, the effervescent tablet composition renders the toilet bowl contents non-toxic and non-corrosive.

In yet another embodiment, the effervescent tablet composition is designed to release carbon dioxide upon contact with water. Carbon dioxide is released as a result of the chemical reaction between sodium bicarbonate salt and citric acid in the presence of water.

In yet another embodiment, a method for concealing or eliminating odorous bathroom smells is described. The method includes the steps of providing a coin-sized effervescent tablet. The effervescent tablet includes quickly dissolvable ingredients, like sodium bicarbonate and added natural oils or powders for emitting a pleasant and odor masking scent. Next, dropping the effervescent tablet into a toilet bowl and dissolving the tablet into toilet bowl water creates an effervescence in the toilet bowl and releases pleasant and natural fragrance for masking unwanted smell and odor.

In yet another embodiment, a process for masking toilet bowl odor is described. The process includes providing a small and discreet effervescent tablet and then unwrapping the effervescent tablet by tearing a wrapper in which the tablet is wrapped. Then, dropping the tablet into a bowl or receptacle of water prior to using the restroom or other area where the fixture may exist. The tablet dissolves in the toilet bowl water for creating effervescence and releasing a pleasant fragrance in the restroom or other room. The tablet of the present invention may also be used for removing or reducing rust stains in the bowl and for ridding fountains and bird paths of bacteria and foul smelling water.

In a further embodiment, the present invention is a tablet for concealing an odor, the tablet comprising an effervescent material, an essential oil, and an acetic acid in an amount greater than the essential oil, wherein the effervescent material is provided in an amount greater than each of the essential oil and acetic acid. The tablet may also include a color changing chemical showing the progress of the tablet as it dissolves.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the effervescent tablet of the present invention for eliminating restroom odors in accordance with the disclosed architecture;

FIG. 2 illustrates an exemplary composition of the toilet bowl odor disguising tablet of the present invention in accordance with the disclosed architecture;

FIG. 3 illustrates yet another exemplary composition of the bowl or receptacle odor disguising or concealing tablet of the present invention in accordance with the disclosed architecture;

FIG. 4 illustrates a flow diagram depicting a process of using the effervescent tablet of the present invention for masking unwanted odor arising from a bowl or water receptacle in accordance with the disclosed architecture;

FIG. 5 illustrates a perspective view showing a user dropping a single effervescent tablet of the present invention in a bowl or other water containing receptacle before or after using the restroom or public area for emitting a pleasant and odor masking scent in accordance with the disclosed architecture;

FIG. 6 illustrates a perspective view showing an effervescent effect caused by the tablet in a water containing bowl or receptacle in accordance with the disclosed architecture;

FIG. 7 illustrates a perspective view showing the tablet being stored and concealed for easy carrying on the go in accordance with the disclosed architecture; and

FIG. 8 illustrates a perspective view showing a consumer package of tablets commercially available for consumers in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for an improved solution that can effectively conceal or cover odors coming from plumbing fixtures in a bathroom or other public spaces. There is also a long-felt need in the art for a fragrance providing device that can be easily and conveniently used in restrooms and specifically with toilets, bidets, sinks, fountains, bird baths and other structures where water is found or where standing water can create a stale or offensive odor. Additionally, there is a long-felt need in the art for a fragrant product that can be easily and conveniently carried by an individual in a pocket or in purse. Moreover, there is a long-felt need in the art for a scent product that is safe for plumbing fixtures such as toilet bowls and septic systems. Further, there is a long-felt need in the art for a product that is easy to use and does not leave any residue after use. Finally, there is a long-felt need in the art for a way that easily conceals or covers restroom odors in public areas like gyms, schools, cafes, restaurants, hotels, convention centers and other areas where a large number of individuals may congregate.

The present invention, in one exemplary embodiment, is a novel method for disguising odorous smells that occur in plumbing or other standing water areas. The method includes the steps of providing a coin-sized effervescent tablet, ranging from 0.7 inches to about 1.2 inches with about 0.8 inches to about 1 inch being preferred. The effervescent tablet includes quickly dissolvable ingredients like sodium bicarbonate, added natural oils or powders, for emitting a pleasant and odor masking scent, and a mild acid. The tablet of the present invention dissolves in between 15 seconds to 30 seconds with about 20 seconds to 25 seconds being preferred, though other durations are also contemplated such as 1-2 minutes. Next, dropping the effervescent tablet into a bowl or receptacle containing water and then dissolving the tablet into the water. The dissolved tablet creates an effervescence in the toilet bowl and releases a pleasant and natural fragrance for masking or concealing unwanted smell and odor in addition to assisting in the reduction of rust in the water containing receptacle and inhibiting bacterial growth.

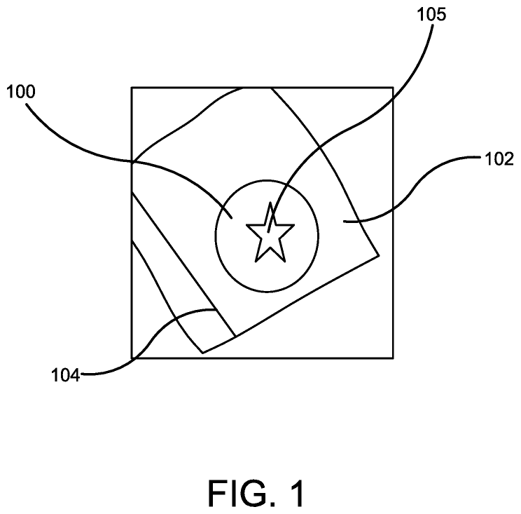

Referring initially to the drawings, FIG. 1 illustrates a perspective view of one potential embodiment of the effervescent tablet 100 of the present invention for eliminating restroom smells and odors in accordance with the disclosed specification. In the present disclosure, “effervescent tablet,”, means a product in the form of a tablet, capsule or the like capable of forming bubbles in a liquid, such as in a water containing receptacle (e.g., toilet, sink, bidet, fountain, bird bath or the like), and capable of liberating carbon dioxide in or out of the liquid. Likewise, “effervescence” means forming bubbles in liquids or liberating carbon dioxide in or out of liquids. More specifically, the tablet 100 is designed as a compact and small tablet that can be easily carried and concealed in purses, waistbands, clothing, wallets, pockets, boots, and even in phone cases. The tablet 100 in the present embodiment has a circular shape, however, can have any geometric shape, including but not limited to, rectangular, square, oval and more. The tablet 100 is wrapped in a moisture proof and easily torn recyclable wrapper 102.

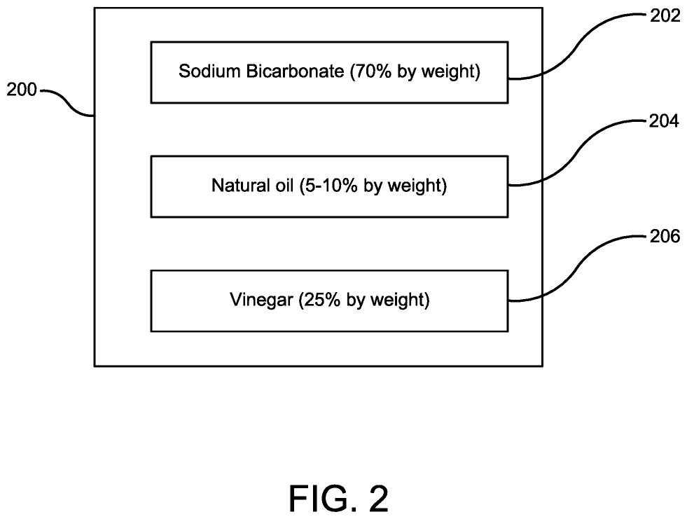

For use, the wrapper 102 is torn off along a pre-marked tear line 104, which may be a perforation line, containing between 3 teeth per 0.32″ to about 17 teeth per 0.32″ creating a hold strength between 10% and 54% and having a light tear strength to a high resistance tear strength. By opening the package along the tear line, the user accesses the tablet 100 and the tablet 100 can be easily dropped into a water containing fixture such as a toilet bowl. The tablet 100 can come in a variety of colors and can be manufactured to release natural fragrance in different variants. The tablet 100 includes natural composition for creating effervescence and releasing natural fragrance as described in FIG. 2. The tablet may also have indicia 105 stamped, printed or otherwise provided on the surface of the tablet.

FIG. 2 illustrates an exemplary composition of the plumbing fixture, standing water, and odor disguising tablet 100 of the present invention in accordance with the disclosed specification. The composition of the tablet 100 is configured to create an effervescence when the tablet 100 is diluted in the water by releasing carbon dioxide. In the present embodiment, the tablet 100 includes sodium bicarbonate 202 preferably 70% by weight of the total composition of the tablet 100. The sodium bicarbonate 202 is configured to absorb bathroom and other odors and also neutralizes acidic odors.

For providing natural fragrance by the tablet 100, the composition 200 has natural or essential oils 204 which may be included in powder or granule form or added to a compressed tablet after formation. The natural or essential oil fragrance is released from the tablet when effervescence takes place inside the plumbing fixture such as a toilet bowl, bidet, sink or other receptacle. The natural or essential oil 204 included in the composition 200 can be one or more of lemon oil, lemongrass oil, rosemary oil, lavender oil, tea tree oil, peppermint, spearmint, citrus and orange oil for providing different fragrances as per desire and preference of users. The natural or essential oil 204 is about 5%-10% by weight of total composition and is instantly released in the restroom or other public bathroom or area of congregation, thus, masking unwanted odors of the restroom, bidet, fixture, and toilet bowl.

For producing effervescence and killing bacteria and pathogens, the composition 200 has vinegar, acetic acid 206. Vinegar 206, such as white vinegar is about 10% to 35% by weight with about 25% by weight being preferred of the total composition of the tablet 100. Sodium bicarbonate 202 reacts with the acetic acid 206 to form carbon dioxide when the tablet 100 is diluted with water. The addition of citrus oil, such as lemon or orange, includes naturally occurring citric acid which, in addition to white vinegar, can assist in reducing rust in the receptacle. In addition, the use of an acetic acid will help in the reduction of bacteria or inhibit the growth of bacteria.

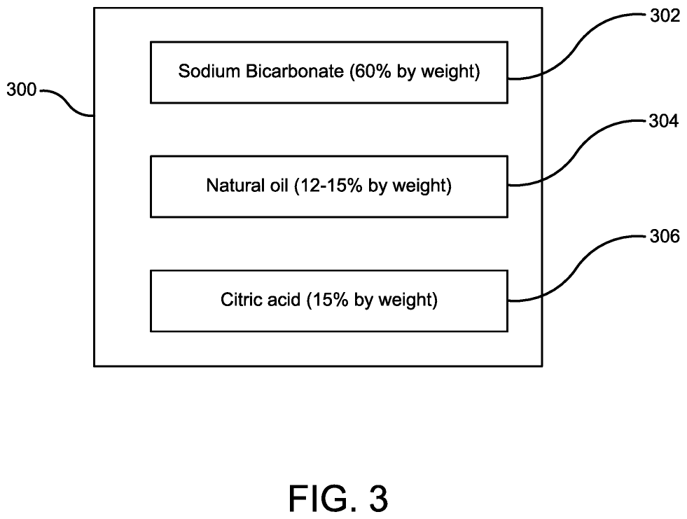

FIG. 3 illustrates yet another exemplary composition of the toilet bowl odor disguising or masking tablet 100 of the present invention in accordance with the disclosed specification. In the present embodiment, the composition 300 includes sodium bicarbonate 302 preferably in the range of 50% to 70% and more preferably around 60% by weight of the total composition of the tablet 100. For providing natural fragrance, the composition 300 has natural oil 304 in powder or granule form or may be added in oil form. The natural oil 304 is in the range of 10% to 20% and more preferably about 12% to 15% by weight of total composition and is instantly released in the restroom or other areas where the public may congregate, thus, masking unwanted odors of the restroom and plumbing fixtures such as a toilet bowl, bidet or other container. The tablet may also include a binder material of between 0.1% to 15% by weight which may include natural and synthetic resins and drying oils between 0.1% to 5% by weight.

For producing effervescence and killing bacteria and pathogens, the composition 300 preferably comprises a citric acid 306 in an amount of between 5% and 20%, and more preferably 15% by weight of the total composition 300.

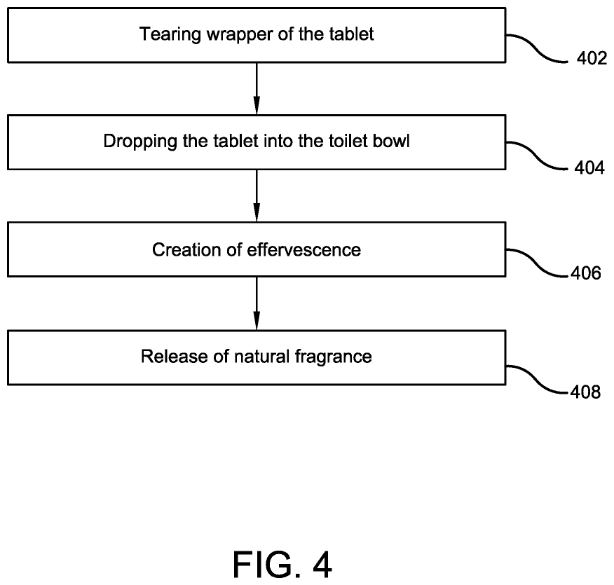

FIG. 4 illustrates a flow diagram depicting a process of using the effervescent tablet of the present invention for masking unwanted odor arising from a plumbing fixture such as a bidet or toilet bowl in accordance with the disclosed specification. As illustrated, initially, a wrapper of an individual tablet is torn, along a tear line or perforation line, by a user intending to mask unwanted odor before or after using the restroom (Step 402). Thereafter, the tablet is dropped into a water holding receptacle such as a toilet bowl or bidet (Step 404). Then, when the tablet is diluted in the water of the plumbing fixture, effervescence is created by the release of carbon dioxide (Step 406). Finally, natural fragrance is released using the natural or essential oil composition of the tablet (Step 408) leaving no residue in the bowl while being safe for the plumbing fixture, sewer or other septic systems.

The tablet 100 can be easily used with any plumbing fixture, such as a bidet or toilet bowl, for eliminating smells and odors and the effect of the tablet 100 is initialized in less than 30 seconds and preferably around 20 seconds.

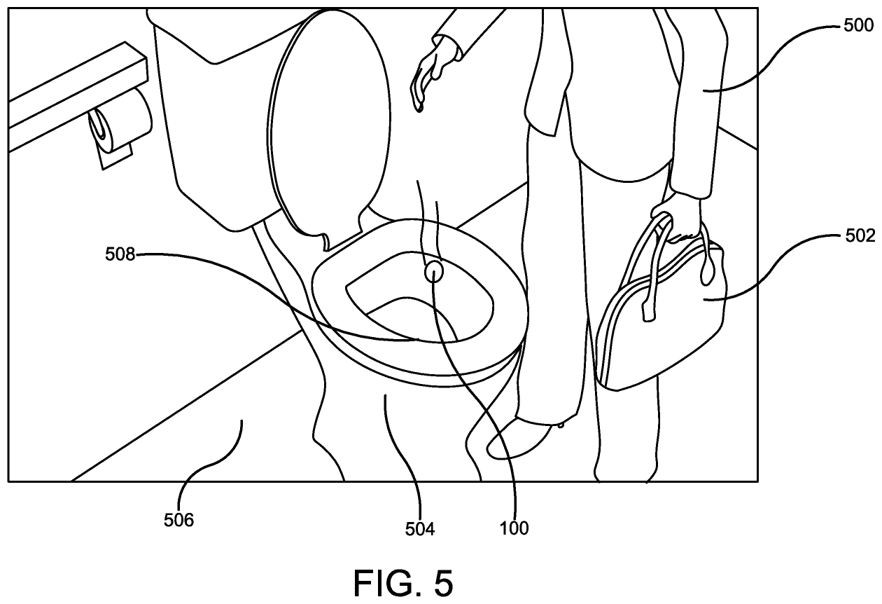

FIG. 5 illustrates a perspective view showing a user dropping a single effervescent tablet 100 of the present invention in a plumbing fixture, such as a bidet or toilet bowl, before or after using the restroom or bathroom, for emitting a pleasant and odor masking scent in accordance with the disclosed specification. The user 500 can take the tablet 100 from a handbag 502 or other area where the tablet may be held, and tear the wrapper as discussed in FIG. 1 for dropping into the plumbing fixture such as a toilet bowl 504 or bidet. The tablet 100 is safe for the toilet bowl 504, the associated sewer or septic system 506. Further, the user 500 can instantly use the plumbing fixture from the toilet bowl 504. The tablet 100 rapidly dissolves in the water 508 of the toilet bowl 504 with no greasy build up.

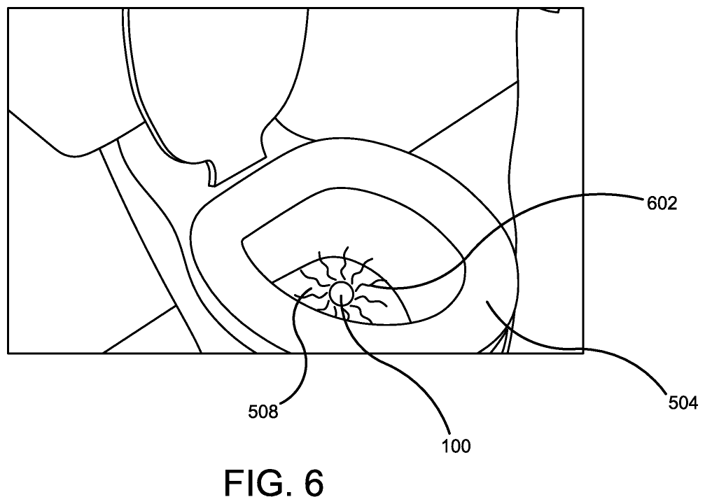

FIG. 6 illustrates a perspective view showing the effervescence effect caused by the tablet 100 in the plumbing fixture, such as the toilet bowl, in accordance with the disclosed specification. As illustrated, when the tablet 100 comes in contact with the water 508 of the plumbing fixture 504, the composition of the tablet releases carbon dioxide in addition to releasing aroma of the natural or essential oils. The sodium bicarbonate and mild acidic constituent of the tablet reacts to form the effervescence 602 and releases carbon dioxide. The aroma of the natural or essential oil masks the unwanted odors of the plumbing fixture, surrounding area and restroom.

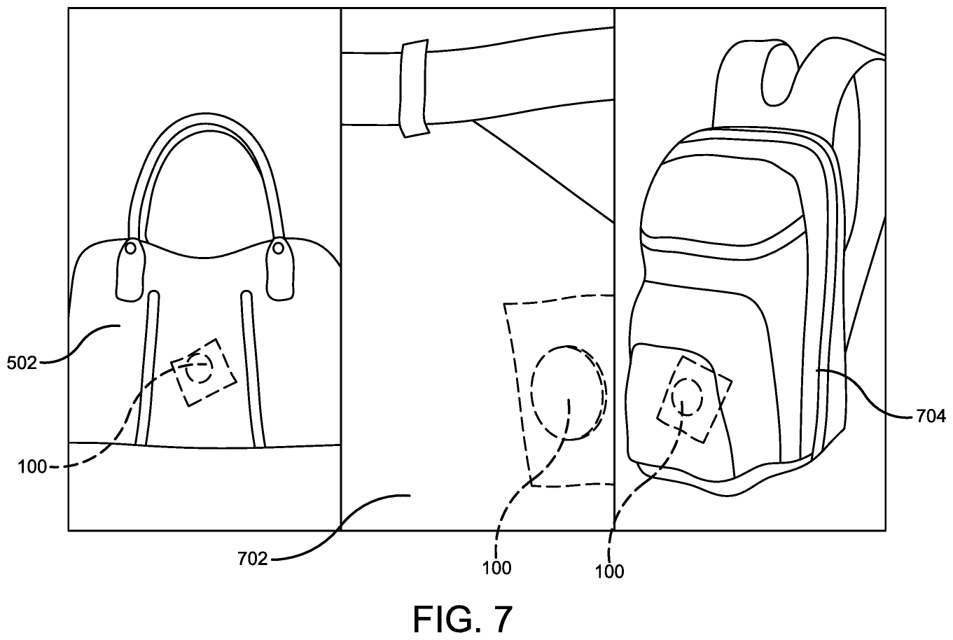

FIG. 7 illustrates a perspective view showing the tablet 100 being stored and concealed for easy carrying on the go in accordance with the disclosed specification. As illustrated, the tablet 100 can be easily stored individually in a handbag 502, a pocket 702 and a backpack 704. The tablet 100 can also be easily stored and concealed in waistbands, clothing, wallets, pockets, boots, and even in phone cases for easy and secure access.

The tablet 100 is great for use when traveling, at the gym, while camping, and especially on first dates or while in close quarters with friends or coworkers.

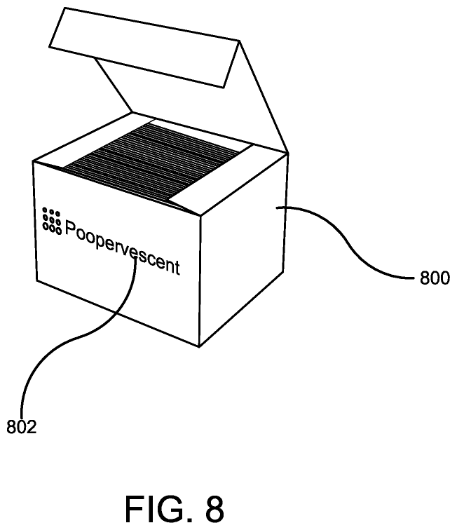

FIG. 8 illustrates a perspective view showing a consumer, or bulk package, of the tablet 100 commercially available for consumers in accordance with the disclosed specification. The effervescent tablet 100 may be packaged in a single use package, which is carried in a purse or a pocket. Alternatively, as shown in the present embodiment, a bulk package 800 is available for storing 20, 50 or 100 tablets and an individual tablet can be taken out of the package 800 for use. The package 800 may have a logo or any other indicia 802 for branding and marketing purposes.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “effervescent tablet”, “tablet”, “toilet bowl odor removing effervescent tablet”, and “toilet bowl odor disguising tablet” are interchangeable and refer to the effervescent tablet 100 of the present invention.

Notwithstanding the forgoing, the effervescent tablet 100 of the present invention can be of any suitable size and configuration as is known in the art without affecting the overall concept of the invention, provided that it accomplishes the above-stated objectives. One of ordinary skill in the art will appreciate that the effervescent tablet 100 as shown in the FIGS. are for illustrative purposes only, and that many other sizes and shapes of the effervescent tablet 100 are well within the scope of the present disclosure. Although the dimensions of the effervescent tablet 100 are important design parameters for user convenience, the effervescent tablet 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A tablet for concealing odor, the tablet comprising:
a tablet formed from an effervescent material;
an essential oil; and
an acetic acid in an amount greater than the essential oil, wherein the effervescent material is provided in an amount greater than each of the essential oil and the acetic acid.

2. The tablet as recited in claim 1, wherein the tablet has a size that ranges from about 0.7 inches to about 1.2 inches.

3. The tablet as recited in claim 1, wherein the tablet has a size that ranges from about 0.8 inches to about 1 inch.

4. The tablet as recited in claim 1 further comprising a binder that constitutes between 0.1 and 15 percent by weight of the tablet and a drying oil that constitutes between 0.1 and 5 percent by weight of the tablet.

5. The tablet as recited in claim 1, wherein the effervescent material is a sodium bicarbonate and is provided in an amount that constitutes between 50 and 70 percent by weight of the tablet.

6. The tablet as recited in claim 1, wherein the essential oil constitutes between 10 and 20 percent by weight of the tablet.

7. The tablet as recited in claim 1, wherein the acetic acid is a white vinegar.

8. The tablet as recited in claim 1, wherein the acetic acid constitutes between 10 and 35 percent by weight of the tablet.

9. The tablet as recited in claim 1, wherein the essential oil is selected from a group consisting of a lemon oil, a lemongrass oil, a rosemary oil, a lavender oil, a tea tree oil, a peppermint, a spearmint, a citrus and an orange oil.

10. The tablet as recited in claim 1, wherein the tablet reduces a rust build up and eliminates a bacterial growth.

11. The tablet as recited in claim 1, wherein the tablet is provided in a round shape.

12. A tablet and package combination for controlling a bathroom odor, the tablet and package combination comprising:
a tablet having between 50 to 70 percent by weight of an effervescent material, a natural oil in an amount between 10 to 20 percent by weight, and a vinegar in an amount between 10 to 30 percent by weight; and
a package sized and configured to hold the table, wherein the package comprises a tear line formed from a plurality of perforations having teeth.

13. A tablet and package combination for controlling bathroom odor as recited in claim 12, wherein the number of teeth in the perforation ranges from between three teeth per 0.32 inches to about seventeen teeth per 0.32 inches.

14. A tablet and package combination for controlling bathroom odor as recited in claim 12, wherein the effervescent material is a sodium bicarbonate.

15. A tablet and package combination for controlling bathroom odor as recited in claim 12, wherein the tablet further comprises at least one of a binder that constitutes between 0.1 and 15 percent by weight of the tablet and a drying oil that constitutes between 0.1 and 5 percent by weight of the tablet.

16. A tablet and package combination for controlling bathroom odor as recited in claim 15, wherein the binder is one of a natural or a synthetic resin.

17. A tablet and package combination for controlling bathroom odor as recited in claim 12, wherein the tablet further comprises a color changing chemical.

18. A tablet and package combination for controlling bathroom odor as recited in claim 12, wherein the natural oil is selected from a group consisting of a lemon oil, a lemongrass oil, a rosemary oil, a lavender oil, a tea tree oil, a peppermint, a spearmint, a citrus and an orange oil.

19. A tablet and package combination for controlling bathroom odor as recited in claim 12, wherein the package is comprised of a moisture proof foil.

20. A deodorizing tablet comprising:
a tablet made from a sodium bicarbonate in an amount ranging between 50 and 70 percent by weight, a vinegar in an amount of 25 percent by weight, and a natural oil in an amount ranging between 10 and 20 percent by weight;
wherein the tablet has a size ranging from between 0.7 and 1.2 inches; and
further wherein the tablet release a fragrance upon dissolving in water contained within a plumbing fixture.
